(19) United States
(12) Patent Application Publication
(10) Pub. No.: US 2026/0137323 A1
(43) Pub. Date: May 21, 2026

(54) EMG ELECTRODE FOR SMALL ANIMALS

(71) Applicant: Albany Medical College, Albany, NY (US)

(72) Inventors: Victor Fenik, Albany, NY (US); Irma Rukhadze, Albany, NY (US)

(73) Assignee: Albany Medical College, Albany, NY (US)

(21) Appl. No.: 19/392,730

(22) Filed: Nov. 18, 2025

Related U.S. Application Data
(60) Provisional application No. 63/722,788, filed on Nov. 20, 2024.

(51) Int. Cl. A61B 5/296; A61B 5/262

(52) U.S. Cl. CPC A61B 5/296; A61B 5/262

(57) ABSTRACT

An electromyogram (EMG) electrode formed from two different length tubes that each enclose one of the two EMG wires and are sized and oriented with respect to each other so that the ends of the two tubes are spaced apart and offset from each other. The ends of the wires are exposed at the ends of the tubes so that positioning of the electrode in the muscle of an animal will allow for long-term EMG recording while reducing tissue damage at the recording site.

### CROSS-REFERENCE TO RELATED APPLICATIONS

The present application claims priority to U.S. Provisional Application No. 63/722,788, filed on Nov. 20, 2024.

### STATEMENT REGARDING FEDERALLY SPONSORED RESEARCH OR DEVELOPMENT

This invention was made with government support under AG065233 awarded by the National Institutes of Health. The government has certain rights in the invention.

### BACKGROUND OF THE INVENTION

### 1. Field of the Invention

The present disclosure relates to electromyography electrodes and, more specifically, to a bipolar electrode for use with the genioglossus muscle.

### 2. Description of the Related Art

An electromyogram (EMG) of the genioglossus (GG) muscle of small behaving animals, such as mice, is usually recorded by inserting two flexible isolated wires into the tongue, which then tunneled under the skin to a connector located on the head of the animal. Unfortunately, the GG recording wires are placed into the tongue blindly simply by aiming at the genioglossus muscle. This approach results in a large variability in the location of the wire tips within the tongue. As a result, the approach leads to inconsistent recording, an increase of data variability, and, thereby, an increase in the number of animals used in experiments. This approach is also undesirable because the movement of the wires and their tips within the highly mobile tongue leads to instability of EMG signal in behaving animals. Finally, moving wires within the tongue may disturb animals, which can affect eating, grooming, etc., and thus considerably change animal behaviors and survival.

### BRIEF SUMMARY OF THE INVENTION

The present invention provides an electrode that solves the problem of stable recording of EMG in small behaving animals, such as mice. The approach of the present invention is also applicable to electrodes used to record EMG of other muscles, such as the diaphragm, and also in electrodes used in larger animals, such as rats. The electrode has a first tube including a first portion extending along a first axis and a second portion extending from the first portion along a second axis and terminating in a first end having a first opening, wherein the first axis and the second axis of the first tube are non-parallel. The electrode has a second tube including a first portion extending along a first axis and a second portion extending from the first portion along a second axis and terminating in second end having a second opening, wherein the first axis and the second axis of the second tube are non-parallel. The first wire extends in the first tube and is exposed by the first opening. A second wire extends in the second tube and is exposed by the second opening. The second tube is shorter than the first tube and oriented relative to the first tube so that the first end and the second end are spaced apart and offset from each other.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a side view of a design for an EMG electrode according to the present invention.

FIG. 2 is a bottom view of a design for an EMG electrode according to the present invention.

FIG. 3 is a front view of a design for an EMG electrode according to the present invention

FIG. 4 is a perspective view of an exemplary EMG electrode according to the present invention.

FIG. 5 is another perspective view of an exemplary EMG electrode according to the present invention.

FIG. 6 is a further perspective view of an exemplary EMG electrode according to the present invention.

FIG. 7 is an additional perspective view of an exemplary EMG electrode according to the present invention.

### DETAILED DESCRIPTION OF THE INVENTION

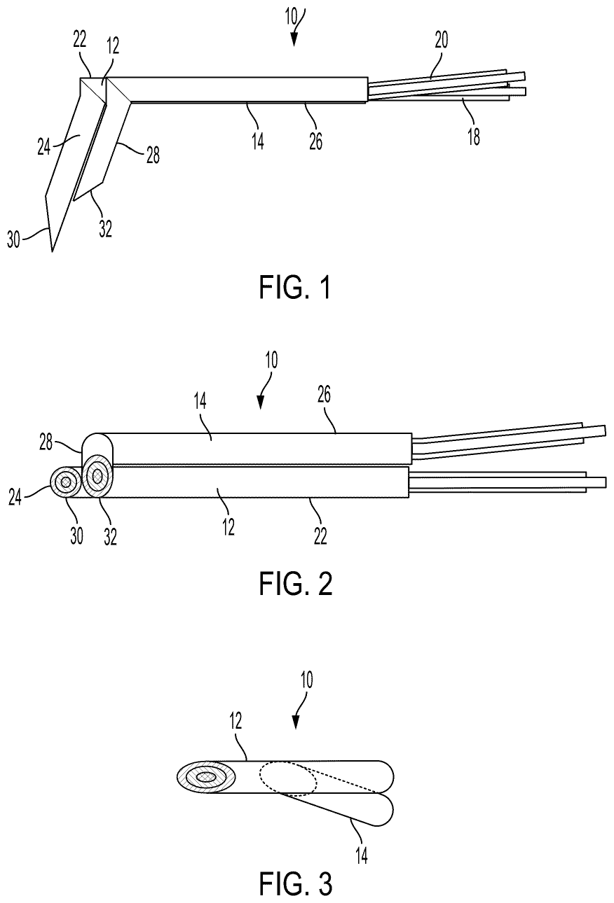

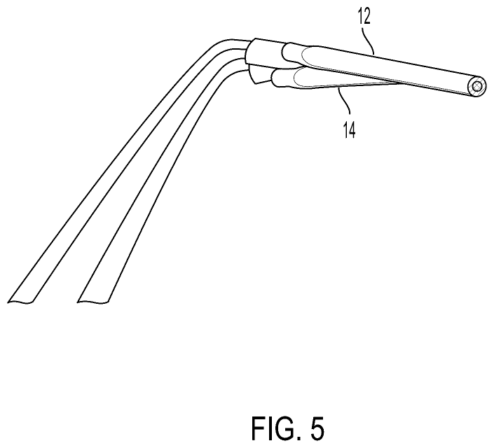

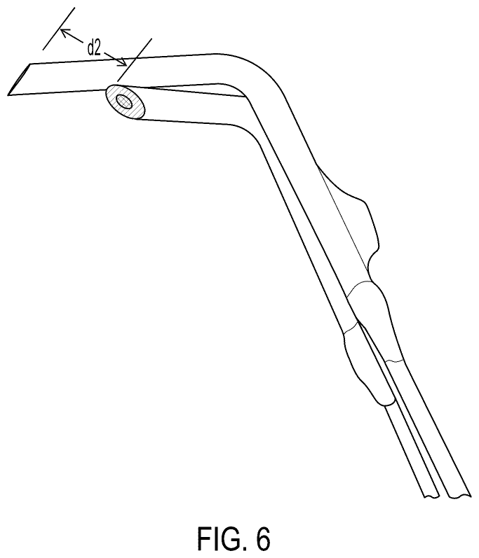

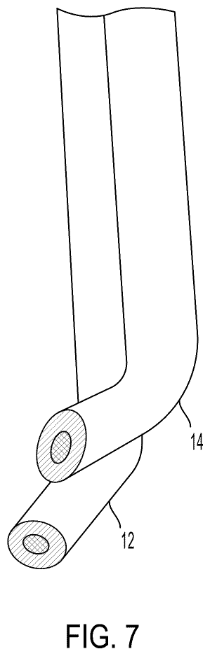

Referring to the figures, wherein like numerals refer to like parts throughout, there is seen in FIG. 1, an electrode 10 according to the present invention that may be used for recording an electromyogram (EMG) of the genioglossus (GG) muscle of small behaving animals. Electrode 10 comprises a first tube 12 and a second tube 14, each of which is coupled to and encloses a first wire 18 and a second wire 20 of an EMG, respectfully. The first tube 12 and second tube 14 are formed from metal and enclose, but are electrically isolated from, first wire 18 and second wire 20. Wires 18 and 20 are preferably insulated stainless-steel wires for bipolar recording of EMG in the genioglossus (GG) muscle or other superficially located muscles, e.g., the diaphragm muscle. Referring to FIGS. 2 and 3, free end 30 and free end 32 expose the recording tips of first wire 18 and second wire 20, respectively. The exposure of first wire 18 and second wire 20 may also be seen in the exemplary electrode of FIGS. 5, 6 and 7.

As seen in FIGS. 1 and 2, first tube 12 is formed from a first portion 22 of electrode 10 that extends longitudinally along a first axis A-A for a first predetermined distance. First tube 12 is further formed by a second portion 24 of electrode 10 that extends from first portion 22 longitudinally along a second axis B-B at an oblique or orthogonal angle thereto so that the first axis A-A and second axis B-B are non-parallel. Second tube 14 is formed by a third portion 26 of electrode 10 that extends for a first predetermined distance longitudinally along a third axis C-C and a fourth portion 28 that extends longitudinally from third portion 26 along a fourth axis D-D at an oblique or orthogonal angle thereto so that the first axis and fourth axis are non-parallel. First portion 22 may be between 2 and 4 millimeters, and is preferably about 3 millimeters. Second portion 24 may be between 2 and 3 millimeters, and is preferably about 2.5 millimeters. Third portion 26 is slightly shorter than first portion 22, and thus may also be between 2 and 4 millimeters provided that it has a length less than that chosen for first portion 22. Fourth portion 28 may be between 1 and 2 millimeters, such as 1.5 millimeters, provided that it has a length shorter than second portion 24.

First tube 12 terminates in a free end 30 that is formed at an angle (a1) relative to the second longitudinal axis of second portion 24. Second tube 14 terminates in a free end 32 that is cut at an angle (a2) relative to the fourth axis of fourth portion 28. The angles of free end 30 and free end 32 relative to the second and fourth axes, respectively, are between 30 and 60 degrees and, preferably 45 degrees. Free end 30 and free end 32 thus form sharp ends that can facilitate insertion of electrode 10 into the GG muscle or other intended location.

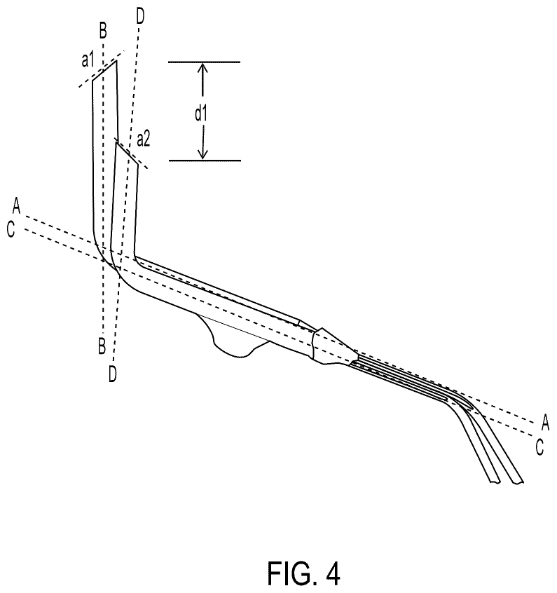

Referring to FIG. 3, second portion 24 of first tube 12 and fourth portion 28 of second tube 14 of electrode are non-planar and extend at an angle relative to each other so that free end 30 and free end 32 are crossed, as seen in FIG. 6. As further seen in FIG. 1 and described above, second portion 24 of first tube 12 is longer than fourth portion 28 of second tube 14. As a result, free end 30 and free end 32 are longitudinally offset from each other, see distance (d1) of FIG. 4, and transversely spaced apart from each other, see distance (d2) of FIG. 6. Distance (d1) may be between 0.9 and 1.1 millimeters, and distance (d2) may be between 0.8 and 1.0 millimeters. This specific positioning of second portion 24 and fourth portion 28 at angle relative to each other with offsetting of free end 30 and free end 32 reduces tissue damage at the recording site when installed in the target muscle of an animal.

The present invention thus provides a miniature bipolar EMG electrode that is suitable for both acute and chronic implantation in animals such as mice to record EMG of superficially located muscles (e.g., genioglossus and diaphragm). For example, electrode 10 may be positioned proximately to the genioglossus (GG) muscle of mice to consistently record a high-quality GG EMG with minimal or no signal contamination from other muscles and without causing damage. Electrode 10 may be used to record EMG of the diaphragm muscle of mice. A low intensity (5 μA) positive current may be passed through electrodes 10 for 1 minute to deposit iron ions and thus enable the verification of the position of electrodes 10 within the tongue. Electrode 10 is useful in basic sleep research to elucidate mechanisms of obstructive sleep apnea (OSA) pathophysiology, drug discovery research, preclinical pharmacological studies to develop OSA pharmacotherapy, and other studies, in which monitoring of genioglossus and/or diaphragmatic muscle activity is required.

The present invention thus provides a miniature bipolar EMG electrode that is suitable for both acute and chronic implantation in animals such as mice to record EMG of superficially located muscles (e.g., genioglossus and diaphragm). For example, electrode 10 may be positioned proximately to the genioglossus (GG) muscle of mice to consistently record a high-quality GG EMG with minimal or no signal contamination from other muscles and without causing damage. Electrode 10 may be used to record EMG of the diaphragm muscle of mice. A low intensity (5 μA) positive current may be passed through electrodes 10 for 1 minute to deposit iron ions and thus enable the verification of the position of electrodes 10 within the tongue. Electrode 10 is useful in basic sleep research to elucidate mechanisms of obstructive sleep apnea (OSA) pathophysiology, drug discovery research, preclinical pharmacological studies to develop OSA pharmacotherapy, and other studies, in which monitoring of genioglossus and/or diaphragmatic muscle activity is required.

What is claimed is:

1. An electrode, comprising:
a first tube formed from a first portion extending along a first axis and a second portion extending from the first portion along a second axis and terminating in a first end having a first opening, wherein the first axis and the second axis of the first tube are non-parallel;
a second tube formed from a third portion extending along a third axis and a fourth portion extending from the third portion along a fourth axis and terminating in second end having a second opening;
a first wire extending through the first tube and exposed by the first opening; and
a second wire extending through the second tube and exposed by the second opening;
wherein the second portion of the first tube is shorter than the third portion of the second tube; and
wherein the first tube is fixed adjacently to the second tube with the first portion and the third portion extending in a common plane and the second portion and the fourth portion extending in different planes so that the first opening is spaced apart from the second opening.

2. The electrode of claim 1, wherein the first opening extends transversely across the first end at a first angle relative to the second axis.

3. The electrode of claim 2, wherein the first angle is between 30 and 60 degrees.

4. The electrode of claim 3, wherein the second opening extends transversely across the second end at a second angle relative to the fourth axis.

5. The electrode of claim 4, wherein the second angle is 30 and 60 degrees.

6. The electrode of claim 1, wherein the third portion is shorter than the first portion.

7. The electrode of claim 6, wherein the fourth portion is shorter than the second portion.

8. The electrode of claim 1, wherein the first opening is offset from the second opening by between 0.9 millimeters and 1.1 millimeters and spaced apart from the second opening by between 0.8 millimeters and 1.0 millimeter.

9. The electrode of claim 8, wherein the second portion has a length of between two and three millimeters.

10. The electrode of claim 9, wherein the fourth portion has a length of between one and two millimeters.

11. A method of performing an electromyogram of a target muscle, comprising the steps of:
providing an electrode comprises of a first tube having a first portion extending along a first axis and a second portion extending from the first portion along a second axis and terminating in a first end having a first opening, wherein the first axis and the second axis of the first tube are non-parallel, a second tube having a third portion extending along a third axis and a fourth portion extending from the third portion along a fourth axis and terminating in second end having a second opening, a first wire extending through the first tube and exposed by the first opening, and a second wire extending through the second tube and exposed by the second opening, wherein the second portion of the first tube is shorter than the third portion of the second tube, and wherein the first tube is fixed adjacently to the second tube with the first portion and the third portion extending in a common plane and the second portion and the fourth portion extending in different planes so that the first opening is spaced apart from the second opening;
positioning the electrode in the target muscle; and
using the electrode to detect electrical activity of the target muscle.

12. The method of claim 11, wherein the first opening extends transversely across the first end at a first angle relative to the second axis.

13. The method of claim 12, wherein the first angle is between 30 and 60 degrees.

14. The method of claim 13, wherein the second opening extends transversely across the second end at a second angle relative to the fourth axis.

15. The method of claim 14, wherein the second angle is 30 and 60 degrees.

16. The method of claim 11, wherein the third portion is shorter than the first portion.

17. The method of claim 16, wherein the fourth portion is shorter than the second portion.

18. The method of claim 11, wherein the first opening is offset from the second opening by 0.9 millimeters and 1.1 millimeters and spaced apart from the second opening by between 0.8 millimeters and 1.0 millimeter.

19. The method of claim 18, wherein the second portion has a length of between two and three millimeters.

20. The method of claim 19, wherein the fourth portion has a length of between one and two millimeters.
